(19) United States
(12) Patent Application Publication
(10) Pub. No.: US 2003/0130314 A1
(43) Pub. Date: Jul. 10, 2003

(54) Analgesic delivery systems and methods of use

(76) Inventor: Pascal Druzgala, Santa Rosa, CA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/321,410

(22) Filed: Dec. 17, 2002

Related U.S. Application Data
(60) Provisional application No. 60/341,743, filed on Dec. 17, 2001.

(51) Int. Cl. A61K 31/445

(52) U.S. Cl. 514/317

(57) ABSTRACT

Novel and advantageous delivery systems for the delivery of analgesic compounds and compositions to patients who are suffering from intractable pain are provided. In some embodiments, these patients already are under the background influence of an opioid agonist. The subject invention also provides methods of managing pain comprising the administration of analgesic compounds via transdermal or transmucosal delivery routes.

### CROSS-REFERENCE TO RELATED APPLICATION

This application claims the benefit of provisional patent application Serial No. 60/341,743, filed Dec. 17, 2001, which is hereby incorporated by reference in its entirety.

### BACKGROUND OF INVENTION

Pain management is an area of great interest in the medical community. Management of pain, and particularly chronic pain, is complex and frequently unsuccessful. In many instances patients enduring chronic or acute pain are under treatment with opioid-based analgesics. The first line of treatment usually involves administration of μ-opioid agonists, e.g., narcotics such as morphine. While it is possible to manage pain in this manner, it is sometimes necessary to provide additional medications for the control of pain.

For example, some patients experience “breakthrough pain”. Typically, patients experiencing breakthrough pain are already under the background influence of opioid therapy for severe pain (for example, terminally ill cancer patients or patients who have experienced major surgery). These patients, even under the background influence of an opioid, suffer episodes of extreme pain when they are moved or when their dressings are changed.

Because of the challenges and complexities of the relevant physiological mechanisms, pain management often involves administration of multiple drugs, such as narcotics, agonist-antagonist agents, butorphanols, benzodiazepines, GABA stimulators, barbiturates, barbiturate-like drugs, orally, e.g., in a pill or liquid formulation, or by i.v. or i.m. injection. Opioid agonists and antagonists may be combined. Thus, a combination of drugs can have offsetting effects. More problematic is the possibility of adverse side effects, particularly gastric distress that accompanies oral administration, or the fear that injections can inspire.

For individuals with chronic pain, there are extensive efforts to identify compounds and methods of providing sustained delivery of analgesics. See, for example, U.S. Pat. No. 6,231,886. Prolonged analgesia is considered to be particulary desirable in patients suffering from moderate to severe pain, such as cancer patients. Available oral preparations provide a duration of effect lasting such that a drug may only have to be administered to a patient one to three times a day. For example, morphine, which has been considered to be the prototypic opioid analgesic, has been formulated into twice-daily, oral controlled release formulations.

Another approach to sustained delivery of a therapeutically active agent are transdermal delivery systems, such as transdermal patches. Generally, transdermal patches contain a therapeutically active agent (e.g., an opioid analgesic), a reservoir or matrix containing the opioid or other active ingredient(s) and an adhesive which allows the transdermal device to adhere to the skin, allowing for the passage of the active agent from the device through the skin of the patient. Once the active agent has penetrated the skin layer, the drug is absorbed into the blood stream where it can exert a desired pharmacotherapeutic effect, such as analgesia

Chronic pain management is an area where new treatments are urgently needed. For example, there is a significant increase in the prevalence and number of cancer deaths worldwide. Pain occurs in more than 80% of cancer patients before death. The World Health Organization has declared pain a world medical emergency. As a result, the use of opioid analgesics has increased worldwide. Fentanyl is an opioid analgesic commonly used in chronic pain management.

Efforts to achieve quicker and more convenient methods of drug delivery have involved the development of nasal and pulmonary delivery mechanisms. These delivery mechanisms have been available for a select number of pharmaceutical agents. For example, aerosol delivery systems with various inhalation-actuated aerosol-dispensing devices have been employed for treatment of asthma, and recently they have been investigating for delivery of insulin. Such devices are breath-activated and designed for delivery to the pulmonary system. See, e.g., U.S. Pat. No. 5,544,646 to Lloyd et al., “Systems for the Intrapulmonary Delivery of Aerosolized Aqueous Formulations”; U.S. Pat. No. 5,320,094 to Laube, “Method of Administering Insulin”; and U.S. Pat. No. 4,648,393 to Landis et al., “Breath Activated Medication Spray”, all of which are incorporated herein.

There remains a need for improved formulations and methods for delivering analgesic agents to patients.

### BRIEF SUMMARY

The subject invention provides novel and advantageous systems for the delivery of analgesic compounds and compositions to patients who are suffering from intractable pain. Specifically exemplified herein are materials and methods for alleviating breakthrough pain such as that which is often experienced by cancer patients who are undergoing chemotherapy.

The subject invention addresses the shortcomings of previous treatments by alleviating short, painful episodes with a small dose of an ultra-short acting opioid administered by a rapid transdermal route or by a transmucosal delivery system. The drugs can be administered by, for example, iontophoresis, ballistics, or via nasal, pulmonary, or sublingual routes.

In specific embodiments of the subject invention, the methods of the subject invention are applied to patients who are already under the background influence of an opioid agonist.

Examples of pain that may be treated according to the subject invention include, and are not limited to, cancer pain, postoperative pain, nociceptive pain (viceral and/or somatic), neuropathic pain (peripheral, central, and/or sympathetic-mediated), and psychogenic (somatization disorders, psychogenic pain, hypochondriasis, and/or specific pain diagnoses (with or without organic contribution).

Thus, the subject invention advantageously provides new methods for the management or treatment of pain that comprise providing a supplemental (or “add-on”) analgesic for pain management.

### DETAILED DISCLOSURE

The subject invention provides novel and advantageous delivery systems for the delivery of analgesic compounds and compositions to patients who are suffering from intractable pain. In specific embodiments of the subject invention, these patients already are under the background influence of an opioid agonist.

In embodiments specifically exemplified herein, the subject invention provides for the delivery of compounds and compositions comprising Formula I.

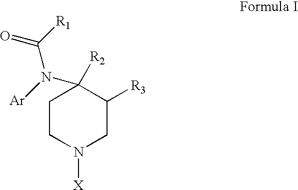

wherein Ar is an aromatic ring optionally mono-, di-, or tri-substituted with halogen, hydroxyl, hydroxymethyl, carbalkoxy, lower alkyl, lower alkoxy, or trifluoromethyl;

R₁ is lower alkyl or lower alkoxy-lower alkyl;

R₂ is H, lower alkoxycarbonyl, or methoxymethyl;

R₃ is H or CH₃; and

X is alkoxycarbonyl-lower alkyl, lower alkyl-carbonyloxy-lower alkyl, alkenyloxycarbonyl-lower alkyl, (C₁₋₂)alkoxy-(C₁₋₂)alkoxycarbonyl-lower alkyl.

As used herein, the term “optionally substituted” means optionally substituted with one or more of the groups specified, at any available position or positions.

As used herein, reference to “lower” alkyl, alkoxyl, etc. includes groups that have from 1 to 6 carbon atoms. These groups may, optionally, be substituted.

As used herein, reference to a “patient” includes human and other animals. The other animals include other primates and mammals.

As used herein, reference to “rapid” transdermal delivery includes a delivery method that delivers the active ingredient more rapidly than a standard transdermal patch. The standard patch is one that delivers the active ingredient as the result of a concentration gradient. Examples of rapid transdermal delivery include ballistic methods and iontophoresis.

Exemplary compounds useful in the practice of the subject invention are disclosed, for example, in U.S. Pat. No. 5,019,583.

The compounds of Formula I are ultrashort acting μ-opioid agonists that were developed for the specific purpose of creating a state of anesthesia and deep analgesia in patients suffering from extreme pain (for example, in patients undergoing surgery); see, for example, U.S. Pat. Nos. 5,019,583 and 5,466,700, hereby incorporated by reference in their entireties. The usefulness of the compounds for alleviating severe pain is known as is their delivery by oral, transdermal, rectal, and parenteral delivery systems.

However, it has, unexpectedly, been found that these compounds, when administered transmucosally, or transdermally by, for example, iontophoresis, have special therapeutic applications in pain management that cannot be achieved via administration by oral, rectal, or regular transdermal systems. Thus, the methods of managing pain, or breakthrough pain, are not accessible by simply using the teaching of U.S. Pat. No. 5,019,583.

In a specific embodiment, the subject invention comprises the delivery of remifentanil (Ultiva®), using iontophoresis or a transmucosal delivery system, and treating breakthrough pain in patients already under background pain management by another opioid agonist (for example, fentanyl, sufentanyl, alfentanyl, morphine, morphine sulfate, or morphine glucuronide).

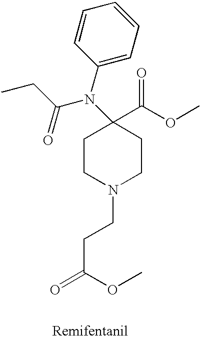

In particular, the methods and delivery systems of the invention are especially efficacious in the treatment of breakthrough pain in patients, or in the general area of pain management. Additionally, the disclosed therapeutic applications are not practical if the compounds are administered parenterally.

It is important to make the distinction between oral delivery systems, such as those disclosed in U.S. Pat. No. 5,019,583, and transmucosal delivery systems such as pulmonary, nasal, and sublingual. Oral systems involve passage through the digestive tract; drugs that are delivered by oral delivery systems undergo the influence of first-pass effect through the liver. However, transmucosally administered drugs (e.g., those administered via nasal, pulmonary, or sublingual routes) do not undergo first-pass metabolism and their onset of action is much faster.

It is also important to make a distinction between regular transdermal application and rapid transdermal delivery including, for example, iontophoresis. Iontophoresis is a special case of transdermal administration where the drug is driven through the skin by the influence of an electric field, such as, for example, in the Alza's E-Trans® system or other patient-controlled apparatus or PCA. Typical transdermal systems, on the other hand, are regular dermal patches from which the drug is released and is driven through the skin solely under the influence of concentration gradients. As a result, iontophoresis is a much faster and a much more reliable way to deliver drugs through the skin than regular patches.

In addition, there is greater inter-individual variability in administering drugs through the skin by regular transdermal systems than by iontophoresis. This variability is acceptable for continuous administration of a drug such as with a 3-day patch; however, such variability is a problem when an acute dose must be administered rapidly and precisely. This is especially true of a potent drug such as the opioid agonists discussed herein. Thus, the subject invention provides for the use of compounds of, for example, Formula I, and of remifentanil in particular, in transmucosal delivery systems, or by rapid transdermal delivery, for important therapeutic purposes that have not been previously taught.

Examples of pain that may be treated according to the subject invention include, and are not limited to, cancer pain, postoperative pain, nociceptive pain (viceral and/or somatic), neuropathic pain (peripheral, central, and/or sympathetic-mediated), and psychogenic (somatization disorders, psychogenic pain, hypochondriasis, and/or specific pain diagnoses (with or without organic contribution). Thus, the subject invention provides new methods for the management or treatment of pain that comprise providing a supplemental (or “add-on”) analgesic for pain management.

Another example of pain suitable for treatment according to the subject invention is breakthrough pain. Typically, patients experiencing breakthrough pain are already under the background influence of opioid therapy for severe pain (for example, terminally ill cancer patients). These patients, even under the background influence of an opioid agonist, suffer episodes of extreme pain when they need to be moved or when their dressings are changed.

These short and painful episodes can be adequately covered by a small dose of an analgesic, such as an ultra-short acting opioid, administered according to this invention in amounts effective to control or manage pain. This administration can be efficiently achieved by iontophoresis or by transmucosal delivery systems; however, pain management by the compounds of Formula I is not possible if the drugs are administered by oral dosage forms, or rectally. Furthermore, it is not practical parenterally to administer these drugs.

The invention provides for the delivery and administration of analgesics by rapid transdermal delivery or by a transmucosal delivery system. Thus, the subject invention also provides transdermal delivery devices and transmucosal delivery systems that are suitable for the management of pain. In this embodiment of the invention, the transmucosal or transdermal delivery systems contain small amounts of analgesics (e.g., opioids) in amounts effective for the control of pain. The devices/delivery systems can be patient or physician/healthcare provider controlled.

Administration of analgesics by rapid transdermal delivery and transmucosal administration are faster and more reliable than by other means because inter-individual variability is minimized and hepatic first-pass metabolism is avoided. Examples of transmucosal systems are pulmonary delivery systems, nasal sprays, and sublingual systems. It is, therefore, an object of this invention to deliver the compounds of, for example, Formula I by iontophoresis (or, for example, transdermal ballistics) or by transmucosal application (e.g., pulmonary, nasal, or sublingual administration).

One mode of transmucosal administration is sublingual, where compounds can be formulated in sublingual sprays/liquids, oromucosal sprays/liquids, or a sublingual tablet. Methods of formulating sublingual sprays, liquids, and tablets are well-known in the art. Likewise, methods of formulating oromucosal sprays and liquids are also well-known in the art. The sublingual tablets typically have rapid dissolution times (minutes) so that the entirety of the dose is rapidly absorbed. It is, therefore, another object of this invention to deliver compounds of, for example, Formula I using a sublingual form (e.g., solid, liquid, or sprayable), for the purpose of treating breakthrough pain in patients who already are on a background pain suppressant, such as another opioid agonist. Exemplary background opioid agonists include, but are not limited to, fentanyl, sufentanyl, alfentanyl, morphine, morphine sulfate, or morphine glucuronide.

The compounds can also be provided in their salt form. Thus, the invention includes pharmaceutically acceptable salts, for example acid addition salts derived from inorganic or organic acids, such as hydrochlorides, hydrobromides, p-toluenesulfonates, phosphates, sulfates, perchlorates, acetates, trifluororacetates, proprionates, citrates, malonates, succinates, lactates, oxalates, tartrates, and benzoates. Salts may also be derived from bases (organic and inorganic), such as alkali metal salts (e.g., magnesium or calcium salts), or organic amine salts, such as morpholine, piperidine, dimethylamine, or diethylamine salts.

Additional modifications of the compounds disclosed herein can readily be made by those skilled in the art. Thus, analogs and salts of the exemplified compounds are within the scope of the subject invention. With a knowledge of the compounds of the subject invention skilled chemists can use known procedures to synthesize these compounds from available substrates. As used in this application, the term “analogs” refers to compounds which are substantially the same as another compound but which may have been modified by, for example, adding additional side groups. The term “analogs” as used in this application also may refer to compounds which are substantially the same as another compound but which have atomic or molecular substitutions at certain locations in the compound.

Analogs of the exemplified compounds can be readily prepared using commonly known, standard reactions. These standard reactions include, but are not limited to, hydrogenation, methylation, acetylation, and acidification reactions. For example, new salts within the scope of the invention can be made by adding mineral acids, e.g., HCl H₂SO₄, etc., or strong organic acids, e.g., formic, oxalic, etc., in appropriate amounts to form the acid addition salt of the parent compound or its derivative. Also, synthesis type reactions may be used pursuant to known procedures to add or modify various groups in the exemplified compounds to produce other compounds within the scope of the invention.

The compounds of the subject invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulation which can be used in connection with the subject invention. In general, the compositions of the subject invention are formulated such that an effective amount of the bioactive compound(s) is combined with a suitable carrier in order to facilitate effective administration of the composition.

In accordance with the subject invention, pharmaceutical compositions are provided which comprise, as an active ingredient, an effective amount of one or more of the compounds and one or more non-toxic, pharmaceutically acceptable carriers or diluents. Examples of such carriers for use in the invention include ethanol, dimethyl sulfoxide, glycerol, silica, alumina, starch, and equivalent carriers and diluents.

Further, acceptable carriers can be either solid or liquid. A solid carrier can be one or more substances that may act as diluents, flavoring agents, solubilizers, lubricants, suspending agents, binders, preservatives, tablet disintegrating agents or encapsulating materials.

The disclosed pharmaceutical compositions may be subdivided into unit doses containing appropriate quantities of the active component. The unit dosage form can be a packaged preparation.

All patents, patent applications, provisional applications, and publications referred to or cited herein are incorporated by reference in their entirety, including all figures and tables, to the extent they are not inconsistent with the explicit teachings of this specification.

It should be understood that the embodiments described herein are for illustrative purposes only and that various modifications or changes in light thereof will be suggested to persons skilled in the art and are to be included within the spirit and purview of this application.

1. A method for relieving pain in a patient, wherein said method comprises administering to a patient in need of pain relief an analgesic compound, or a salt or analog of said compound, wherein said compound has the following formula: 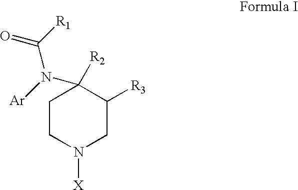
wherein Ar is an aromatic ring optionally mono-, di-, or tri-substituted with halogen, hydroxyl, hydroxymethyl, carbalkoxy, lower alkyl, lower alkoxy, or trifluoromethyl;
R₁ is lower alkyl or lower alkoxy-lower alkyl;
R₂ is H, lower alkoxycarbonyl, or methoxymethyl;
R₃ is H or CH₃; and
X is alkoxycarbonyl-lower alkyl, lower alkyl-carbonyloxy-lower alkyl, alkenyloxycarbonyl-lower alkyl, (C₁₋₂)alkoxy-(C₁₋₂)alkoxycarbonyl-lower alkyl; wherein said delivery is transmucosal or by rapid transdermal delivery.

2. The method, according to claim 1, which comprises administering a compound having the following formula: 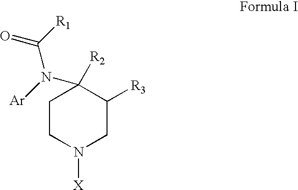
wherein Ar is an aromatic ring optionally mono-, di-, or tri-substituted with halogen, hydroxyl, hydroxymethyl, carbalkoxy, lower alkyl, lower alkoxy, or trifluoromethyl;
R₁ is lower alkyl or lower alkoxy-lower alkyl;
R₂ is H, lower alkoxycarbonyl, or methoxymethyl;
R₃ is H or CH₃; and
X is alkoxycarbonyl-lower alkyl, lower alkyl-carbonyloxy-lower alkyl, alkenyloxycarbonyl-lower alkyl, (C₁₋₂)alkoxy-(C₁₋₂)alkoxycarbonyl-lower alkyl; wherein said delivery is transmucosal or by rapid transdermal delivery.

3. The method, according to claim 1, wherein said patient has received background treatment with a compound selected from the group consisting of fentanyl, sufentanyl, alfentanyl, morphine, morphine sulfate, and morphine glucuronide.

4. The method, according to claim 1, wherein said compound is remifentinil.

5. The method, according to claim 1, wherein said delivery is done transmucosally or by iontophoresis.

6. The method, according to claim 1, wherein said patient is a human.

7. The method, according to claim 6, wherein said patient has previously been, or is currently being, treated with an opiod agonist.

8. The method, according to claim 1, wherein said patient is an animal.

9. The method, according to claim 1, wherein said pain is selected from the group consisting of cancer pain, postoperative pain, nociceptive pain, neuropathic pain and psychogenic pain.

10. The method, according to claim 1, wherein said administration is done by a route selected from the group consisting of iontophoresis, ballistic, nasal, pulmonary and sublingual.

11. A device for delivering an analgesic by rapid transdermal delivery or transmucosal delivery.

12. The device, according to claim 11, comprising an analgesic compound, or a salt or analog of said compound, wherein said compound has the following formula: 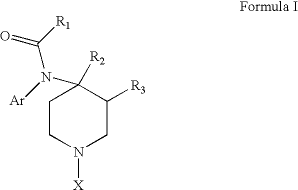
wherein Ar is an aromatic ring optionally mono-, di-, or tri-substituted with halogen, hydroxyl, hydroxymethyl, carbalkoxy, lower alkyl, lower alkoxy, or trifluoromethyl;
R₁ is lower alkyl or lower alkoxy-lower alkyl;
R₂ is H, lower alkoxycarbonyl, or methoxymethyl;
R₃ is H or CH₃; and
X is alkoxycarbonyl-lower alkyl, lower alkyl-carbonyloxy-lower alkyl, alkenyloxycarbonyl-lower alkyl, (C₁₋₂)alkoxy-(C₁₋₂)alkoxycarbonyl-lower alkyl.
